(19) United States
(12) Patent Application Publication
(10) Pub. No.: US 2020/0253676 A1
(43) Pub. Date: Aug. 13, 2020

(54) ROBOTICALLY-OPERATED UTERINE MANIPULATORS

(71) Applicant: Covidien LP, Mansfield, MA (US)

(72) Inventor: Zachary TRAINA, Verona, NJ (US)

(21) Appl. No.: 16/777,989

(22) Filed: Jan. 31, 2020

Related U.S. Application Data
(60) Provisional application No. 62/804,405, filed on Feb. 12, 2019.

(51) Int. Cl. A61B 34/00; A61B 90/50; A61B 17/42

(52) U.S. Cl. CPC A61B 34/71; A61B 2017/00477; A61B 17/42; A61B 90/50

(57) ABSTRACT

A robotically-operated uterine manipulator includes an articulation assembly having a driven member configured to be operably coupled to a first drive member of an instrument drive unit. A nut of the articulation assembly is threadedly coupled to the driven member. A cable of the articulation assembly has a proximal end portion coupled to the nut, and a distal end portion coupled to a tip hub. The cable is configured to translate with the nut and relative to the driven member in response to a rotation of the driven member to articulate the tip hub.

### CROSS-REFERENCE TO RELATED APPLICATIONS

The present application claims the benefit of and priority to U.S. Provisional Patent Application Ser. No. 62/804,405, filed on Feb. 12, 2019, the entire content of which is incorporated herein by reference.

### BACKGROUND

Uterine manipulators are medical instruments used for manipulating (e.g., moving or repositioning) a patient's uterus during medical procedures. Such procedures include laparoscopic gynecologic surgery, e.g., total laparoscopic hysterectomy (TLH) surgery. Uterine manipulators typically include a proximal portion that remains external to the patient's body during use and a distal portion that is inserted into the patient's body. The proximal portion typically provides for manipulation of the instrument during use. The distal portion often includes a tip sized to be inserted into and/or engage a uterus. The distal portion of the instrument is advanced through the vaginal cavity and into the uterus. With the distal portion inserted within a uterus, the uterus can be manipulated through surgeon- or physician-controlled movements of the proximal portion. Following completion of a procedure, the instrument may be removed from the patient's body via the vaginal cavity.

### SUMMARY

In one aspect of the present disclosure, a robotically-operated uterine manipulator is provided and includes a housing configured to be coupled to an instrument drive unit, a shaft extending distally from the housing, a tip hub pivotably coupled to a distal end portion of the shaft, and an articulation assembly. The articulation assembly includes a first driven member, a first nut, and a first cable. The first driven member is rotationally supported in the housing and has a proximal end portion configured to be operably coupled to a first drive member of the instrument drive unit. The first nut is threadedly coupled to the first driven member, such that the first nut translates along the first driven member upon rotation of the first driven member. The first cable has a proximal end portion fixed to the first nut, and a distal end portion coupled to the tip hub. The first cable is configured to translate with the first nut and relative to the first driven member in response to a rotation of the first driven member to articulate the tip hub relative to the shaft.

In aspects, the articulation assembly may include a second driven member, a second nut, and a second cable. The second driven member may be rotationally supported in the housing and have a proximal end portion configured to be operably coupled to a second drive member of the instrument drive unit. The second nut may be threadedly coupled to the second driven member, such that the second nut translates along the second driven member upon rotation of the second driven member. The second cable may have a proximal end portion fixed to the second nut, and a distal end portion. The distal end portion of the first cable may be coupled to a first side of the tip hub, and the distal end portion of the second cable may be coupled to a second side of the tip hub.

In some aspects, the uterine manipulator may further include a rotatable collar. The first driven member may have a distal end portion operably coupled to the collar, such that a rotation of the collar rotates the first driven member.

In further aspects, the collar may have a plurality of gear teeth extending from an inner periphery thereof. The distal end portion of the first driven member may have a gear in meshing engagement with the plurality of gear teeth of the collar.

In other aspects, the proximal end portion of the first driven member may have an input coupler configured to be non-rotatably coupled to an output coupler of the instrument drive unit.

In aspects, the input coupler may be accessible from outside of the housing.

In some aspects, the first driven member may be parallel with and offset from a longitudinal axis defined by the shaft.

In another aspect of the disclosure, a robotic surgical assembly is provided and includes an instrument drive unit and a robotically-operated uterine manipulator. The instrument drive unit has a first motor and a first drive member drivingly coupled to the first motor, and the uterine manipulator has a housing, a shaft, a tip hub, a tip member, and an articulation assembly. The housing is configured to be coupled to the instrument drive unit. The shaft extends distally from the housing and the tip hub is pivotably coupled to a distal end portion of the shaft. The tip member is configured to be attached to the tip hub. The articulation assembly includes a first driven member, a first nut, and a first cable. The first driven member is rotationally supported in the housing and has a proximal end portion configured to be operably coupled to the first drive member of the instrument drive unit. The first nut is threadedly coupled to the first driven member, such that the first nut translates along the first driven member upon rotation of the first driven member. The first cable has a proximal end portion fixed to the first nut, and a distal end portion coupled to the tip hub. The first cable is configured to translate with the first nut and relative to the first driven member in response to a rotation of the first driven member to articulate the tip member relative to the shaft.

In aspects, the uterine manipulator may include a manually-rotatable collar, and the first driven member may have a distal end portion operably coupled to the collar, such that a rotation of the collar rotates the first driven member.

In some aspects, the proximal end portion of the first driven member may have an input coupler configured to be non-rotatably coupled to an output coupler of the first drive member.

In further aspects, the robotic surgical assembly may further include a robotic arm configured to support the instrument drive unit and the uterine manipulator.

In accordance with yet another aspect of the disclosure, a robotically-operated uterine manipulator is provided and includes a housing configured to be coupled to an instrument drive unit, a shaft extending distally from the housing, a tip hub pivotably coupled to a distal end portion of the shaft, first and second driven members, first and second nuts, and first and second cables. The first and second driven members are rotationally supported in the housing and each has a proximal end portion configured to be operably coupled to a respective drive member of the instrument drive unit. The first and second nuts are threadedly coupled to the respective first and second driven members, such that the first and second nuts are independently movable along the respective first and second driven members upon rotation of the first and second driven members. Each of the first and second cables has a proximal end portion coupled to the respective first and second nuts, and a distal end portion coupled to the tip hub. The first and second cables are configured to translate in opposite directions from one another in response to a rotation of the first and second driven members to articulate the tip hub relative to the shaft.

In aspects, the uterine manipulator may further include a rotatable collar, and each of the first and second driven members may have a distal end portion operably coupled to the collar, such that a rotation of the collar rotates the first and second driven members to translate the first and second cables in opposite directions from one another.

In some aspects, the collar may have a plurality of gear teeth extending from an inner periphery thereof, and the distal end portion of each of the first and second driven members may have a gear in meshing engagement with the plurality of gear teeth of the collar.

In further aspects, the proximal end portion of each of the first and second driven members may have an input coupler configured to be non-rotatably coupled to a corresponding output coupler of the instrument drive unit.

In other aspects, the first and second driven members may be parallel with and offset from a longitudinal axis defined by the shaft.

As used herein, the term distal refers to that portion of the described component which is farthest from the user, while the term proximal refers to that portion of the described component which is closest to the user.

As used herein, the term parallel is understood to include relative configurations that are substantially parallel up to about + or −10 degrees from true parallel.

### BRIEF DESCRIPTION OF THE DRAWINGS

The accompanying drawings, which are incorporated in and constitute a part of this specification, illustrate embodiments of the disclosure and, together with a general description of the disclosure given above, and the detailed description of the embodiment(s) given below, serve to explain the principles of the disclosure, wherein:

FIG. 1 is a schematic illustration of a robotic surgical system including a robotic surgical assembly in accordance with the present disclosure;

FIG. 2 is a perspective view illustrating a surgical robotic arm, an instrument drive unit, and a uterine manipulator of the robotic surgical assembly of FIG. 1;

FIG. 3 is a side, perspective view illustrating the uterine manipulator of FIG. 2;

FIG. 4 is another side, perspective view illustrating the uterine manipulator of FIG. 2;

FIG. 5 is a cutaway view illustrating components of an articulation assembly of the uterine manipulator of FIG. 3;

FIG. 6 is a longitudinal cross-sectional view illustrating components of the articulation assembly of the uterine manipulator of FIG. 3;

FIG. 7 is a side, perspective view of a distal portion of the uterine manipulator of FIG. 3; and

FIG. 8 is a side perspective view, with parts removed, of the uterine manipulator of FIG. 7.

### DETAILED DESCRIPTION

Embodiments of the present disclosure will now be described in detail with reference to the drawings, in which like reference numerals designate identical or corresponding elements in each of the several views. In the following description, well-known functions or constructions are not described in detail to avoid obscuring the present disclosure in unnecessary detail.

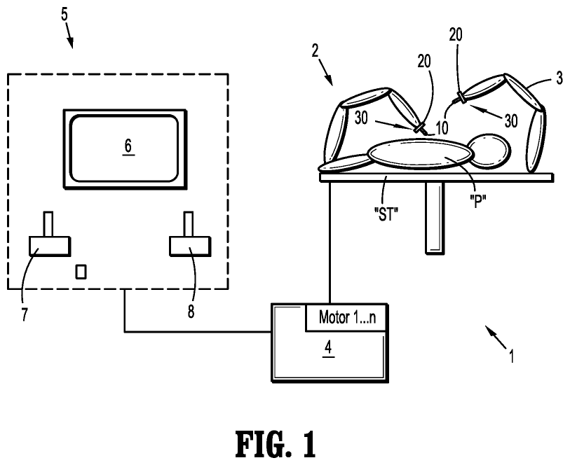

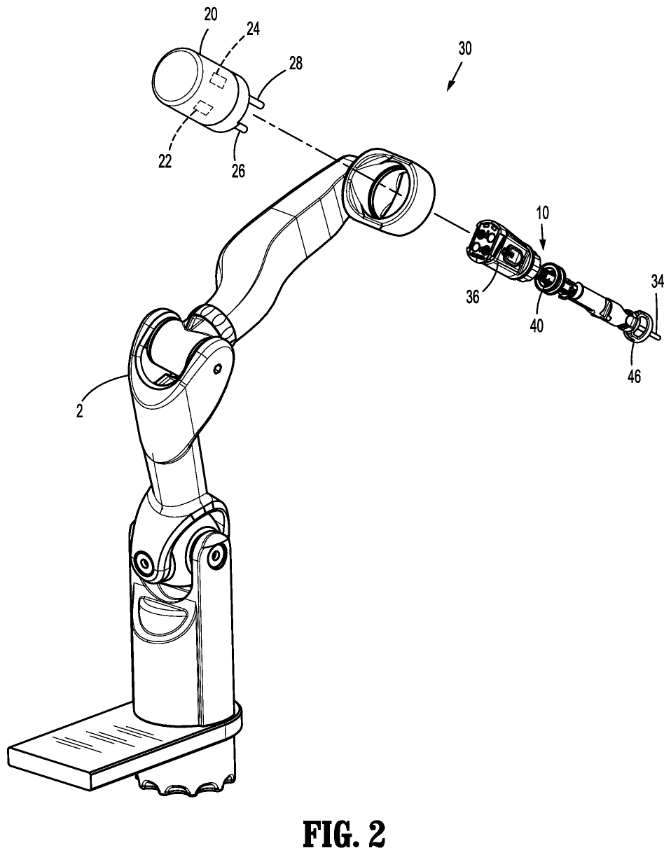

Referring initially to FIGS. 1 and 2, a surgical system, such as, for example, a robotic surgical system 1, generally includes a plurality of surgical robotic arms 2, 3 having an instrument drive unit 20 removably attached thereto; a uterine manipulator 10 removably attached the instrument drive unit 20; a control device 4; and an operating console 5 coupled to the control device 4.

With continued reference to FIG. 1, the operating console 5 includes a display device 6, which is set up in particular to display three-dimensional images, and manual input devices 7, 8, by means of which a person (not shown), for example a surgeon, is able to telemanipulate the robotic arms 2, 3 in a first operating mode, as known in principle to a person skilled in the art. Each of the robotic arms 2, 3 may include a plurality of members, which are connected through joints. The robotic arms 2, 3 may be driven by electric drives (not shown) that are connected to the control device 4. The control device 4 (e.g., a computer) is set up to activate the drives, in particular by means of a computer program, in such a way that the robotic arms 2, 3, their instrument drive units 20, and thus the uterine manipulators 10 execute a desired movement according to a movement defined by means of the manual input devices 7, 8. The control device 4 may also be set up in such a way that it regulates the movement of the robotic arms 2, 3 and/or of the drives.

The robotic surgical system 1 is configured for use on a patient “P” lying on a surgical table “ST” to be treated in a minimally invasive manner by means of a surgical instrument, e.g., the uterine manipulator 10. The robotic surgical system 1 may also include more than two robotic arms 2, 3, the additional robotic arms likewise being connected to the control device 4 and being telemanipulatable by means of the operating console 5. A surgical instrument, for example, the uterine manipulator 10, may also be attached to the additional robotic arm.

The control device 4 may control a plurality of motors (Motor 1 . . . n) with each motor configured to drive a relative rotation of driven members of the uterine manipulator 10 to effect operation and/or movement of components of the uterine manipulator 10. It is contemplated that the control device 4 coordinates the activation of the various motors (Motor 1 . . . n) to coordinate a clockwise or counter-clockwise rotation of drive members of the instrument drive unit 20 in order to coordinate an operation and/or movement of a respective driven member of the uterine manipulator 10.

For a detailed discussion of the construction and operation of a robotic surgical system, reference may be made to U.S. Pat. No. 8,828,023, filed on Nov. 3, 2011, entitled “Medical Workstation,” the entire contents of which are incorporated herein by reference.

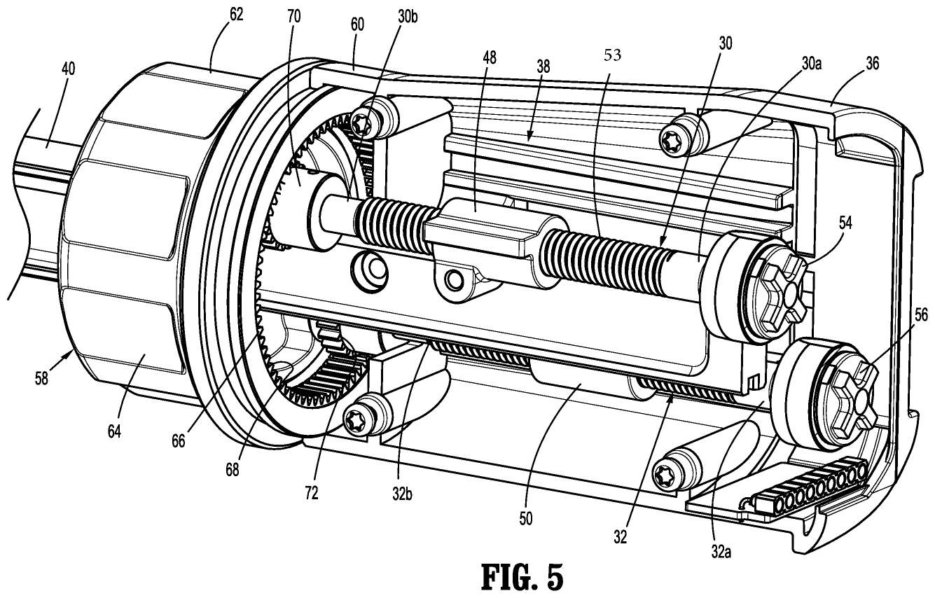

With specific reference to FIG. 2, the robotic surgical system 1 includes a surgical robotic assembly 30, which includes the robotic arm 2, the instrument drive unit 20 configured to be coupled to the robotic arm 2, and the uterine manipulator 10 configured to be coupled to the instrument drive unit 20. The instrument drive unit 20 is configured for powering the uterine manipulator 10 and to transfer power and actuation forces from its motors 22, 24 to driven members 30, 32 (FIG. 5) of the uterine manipulator 10 to ultimately drive movement of components of the uterine manipulator 10, for example, an articulation of a tip member 34 of the uterine manipulator 10. The instrument drive unit 20 includes first and second drive members 26, 28 drivingly coupled to a respective motor 22, 24, such that the drive members 26, 28 are independently rotatable with respect to one another.

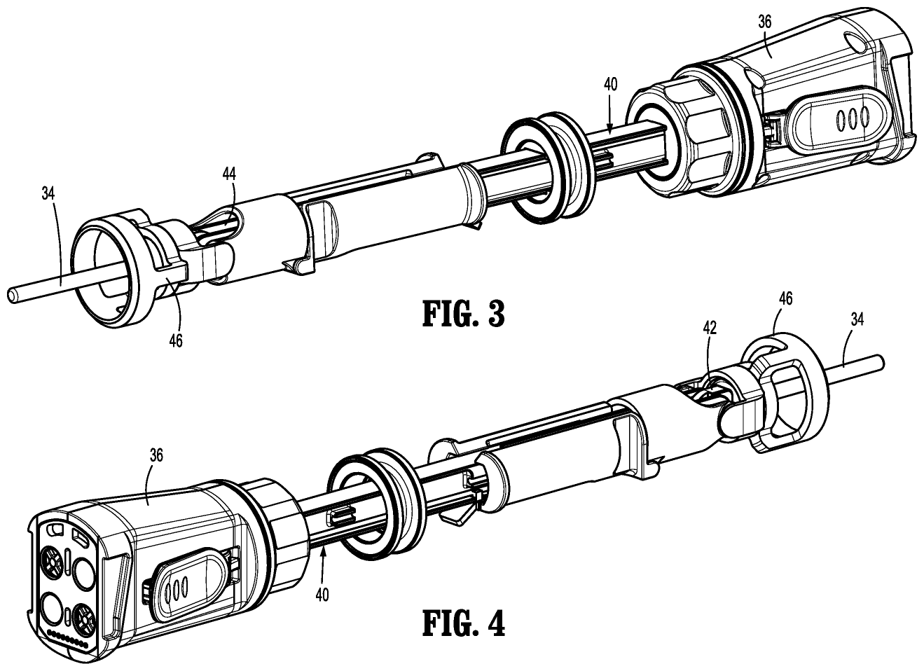

FIGS. 3-8 illustrate the uterine manipulator 10 for use in the robotic surgical system 1 of FIGS. 1-2. The uterine manipulator 10 is configured to be operably coupled to the instrument drive unit 20 and inserted into a vaginal cavity for use in female pelvic surgical procedures, such as a hysterectomy. The uterine manipulator 10 generally includes a housing 36, an articulation assembly 38, a shaft 40 extending distally from the housing 36, a tip hub 42 pivotably coupled to a distal end portion 44 of the shaft 40, the tip member 34 attached to the tip hub 42, and a cervical cup 46 positionable about the shaft 40.

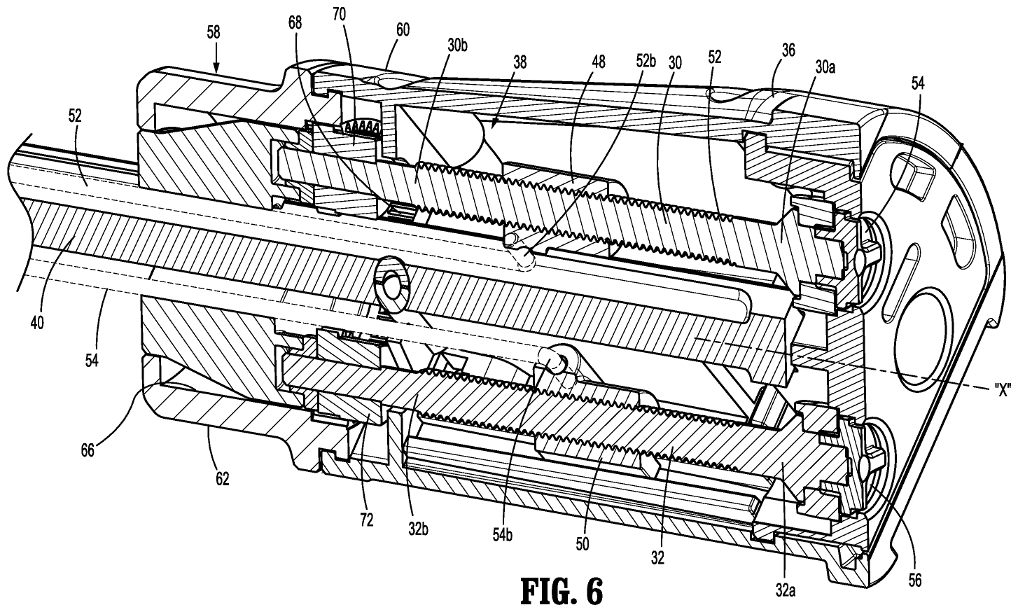

With reference to FIGS. 3-6, the articulation assembly 38 (FIG. 6) is partially disposed within the housing 36 and includes first and second driven members 30, 32 supported in the housing 36, first and second nuts 48, 50 operably coupled to the respective first and second driven members 30, 32, and first and second cables 52, 54 coupled to the respective first and second nuts 48, 50. The first and second driven members 30, 32 may be configured as lead screws having an external threading 53 disposed along their length. Each of the first and second driven members 30, 32 are parallel with and offset from a longitudinal axis “X” defined by the shaft 40 and has a proximal end portion 30a, 32a, and a distal end portion 30b, 32b. The proximal end portion 30a, 32a of each of the driven members 30, 32 has an input coupler 54, 56 (e.g., crossed protrusion) configured to be non-rotatably coupled to a corresponding output coupler of the respective first and second drive members 26, 28 (FIG. 2) of the instrument drive unit 20. As such, when the instrument drive unit 20 is operably coupled to the housing 36 of the uterine manipulator 10, an actuation of the first and second motors 22, 24 of the instrument drive unit 20 drives a rotation of the first and second driven members 30, 32 of the articulation assembly 38.

The uterine manipulator 10 may include a manually-rotatable collar 58 rotatably coupled to a distal end portion 60 of the housing 36 and disposed about the shaft 40. The collar 58 has an outer peripheral surface 62 defining a plurality of surface features 64 configured to assist a clinician in grasping the collar 58. An inner periphery 66 of the collar 58 defines a plurality of gear teeth 68 disposed in a circular array around the shaft 36. The distal end portion 30b, 32b of each of the first and second driven members 30, 32 has a gear 70, 72, such as, for example, a spur gear, attached thereto in meshing engagement with the gear teeth 68 of the collar 58. In aspects, the spur gears 70, 72 may be operably coupled to the gear teeth 68 of the collar 58 via an intermediary gear. In other embodiments, the distal end portion 30b, 32b of each of the first and second driven members 30, 32 may be operably coupled to the collar 58 via any suitable mechanism, such as, for example, frictional engagement.

Due to the engagement between the first and second driven members 30, 32 and the collar 58, a manual rotation of the collar 58 results in a rotation of the first and second driven members 30, 32 about their respective longitudinal axes. It is contemplated that the collar 58 may be used in the instance the instrument drive unit 20 is not attached to the uterine manipulator 10 or if power is otherwise not available.

The first and second nuts 48, 50 of the articulation assembly 38 are threadedly coupled to the threading 53 defined along the respective first and second driven members 30, 32. Each of the first and second nuts 48, 50 may be rotationally restrained by an internal structure of the housing 36 so that rotation of the first and second driven members 30, 32 results in only axial motion of the first and second nuts 48, 50 along the first and second driven members 30, 32.

The first and second cables 52, 54 of the articulation assembly 38 are rigid and maintain a linear shape during actuation of the articulation assembly 38. In some aspects, the cables 52, 54 may be fabricated from a flexible material. The cables 52, 54 each have a proximal end portion 52a, 54a disposed within the housing 36, and a distal end portion 52b , 54b (FIG. 8). The proximal end portion 52a, 54a of each of the cables 52, 54 is fixed to the respective first and second nuts 48, 50 via a pin-slot engagement. In other aspects, the proximal end portion 52a, 54a of each of the cables 52, 54 may be fixed, either directly or indirectly, to the respective first and second nuts 48, 50 via any suitable fastening engagement, such as, for example, adhesives, screws, rivets, knots, or the like.

As best shown in FIG. 8, the distal end portion 52b of the first cable 52 is fixed to a first side 42a of the tip hub 42, and the distal end portion 54b of the second cable 54 is fixed to a second side 42b of the tip hub 42. In this way, a translation of the first cable 52 in a first direction (e.g., a distal direction) coupled with a translation of the second cable 54 in a second direction (e.g., a proximal direction), opposite the first direction, causes an articulation of the tip hub 42, along with the tip member 34, relative to the shaft 40.

With reference to FIGS. 3, 4, 7, and 8, the tip member 34 of the uterine manipulator 10 is attached to the tip hub 42 and has a head portion 76 and an elongate rod 78 extending distally from the head portion 76. In aspects, the tip member 34 may be monolithically formed with the tip hub 42. The head portion 76 may have a distally-facing surface 80 having a frustoconical shape configured to engage the external surface of a cervix. The elongate rod 78 of the tip member 34 may have an elongated, expandable balloon (not shown) for engaging a cervical canal to aid in repositioning of a uterus. The balloon may have a supply line (not shown) extending therefrom configured to be coupled to a source of fluid (e.g., gas or liquid) to selectively expand the balloon.

The cervical cup 46 of the uterine manipulator 10 is configured to capture an endocervix therein and is pivotably coupled to the distal end portion 44 of the shaft 40. The head portion 76 of the tip member 34 is received in an opening 82 defined in the cervical cup 46, which is sized to capture the head portion 76 therein, such that the tip member 34 and the cervical cup 46 articulate together as one unit. The elongate rod 78 of the tip member 34 extends distally from the cervical cup 46 and is centrally disposed therein. The cervical cup 46 may be pivotably coupled to a sleeve 84 that is slidably disposed about and detachably coupled to the shaft 40.

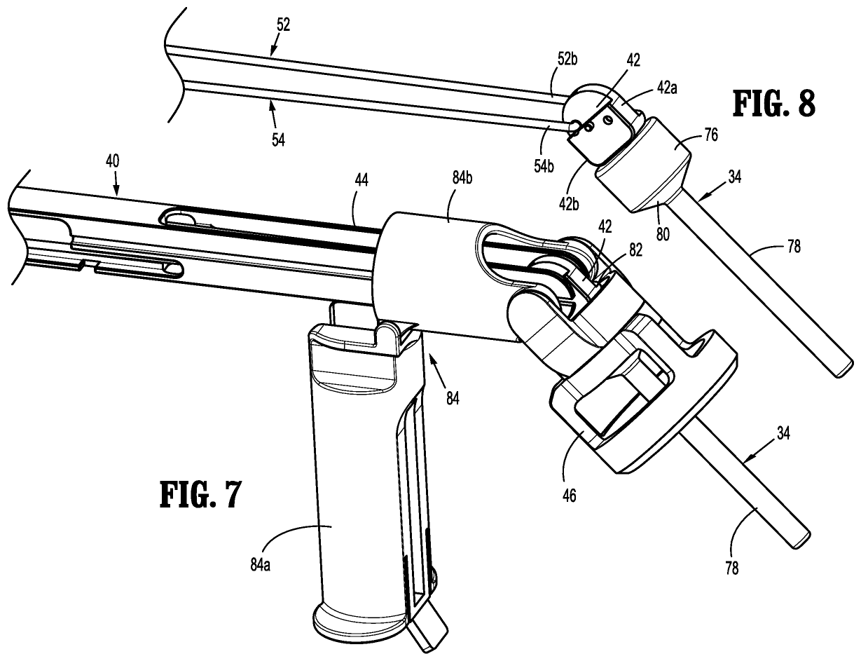

The sleeve 84 includes a distal portion 84b and a proximal portion 84a pivotably coupled to the distal portion 84b . The distal portion 84b of the sleeve 84 is slidable along and relative to the shaft 40 to position the cervical cup 46 about the tip member 34 when ready for use. Upon positioning the distal portion 84b of the sleeve 84 and the attached cervical cup 46 in an in-use position (FIG. 7), the proximal portion 84a of the sleeve 84 may be pivoted from a first state (FIG. 7), in which the proximal portion 84a is disconnected from the shaft 40, to a second state (FIGS. 3 and 4), in which the proximal portion 84a is aligned with the distal portion 84b and is connected to the shaft 40. In the second state, the sleeve 84 and the cervical cup 46 are axially fixed to the shaft 40. It is contemplated that the proximal portion 84a of the sleeve 84 may be releasably engaged to the shaft 40 via a friction fit engagement or any suitable fastening engagement.

The sleeve 84 may have a vaginal occluder balloon (not shown) disposed about the distal portion 84b thereof. The vaginal occluder balloon is expandable within the vagina to facilitate securing the uterine manipulator in a selected position relative to the uterus. The vaginal occluder balloon may have a supply line (not shown) extending therefrom configured to be coupled to a source of fluid (e.g., gas or liquid) for selectively expanding the vaginal occluder balloon.

In operation, a hysterectomy may be performed utilizing the surgical robotic assembly 30 of the present disclosure. During or in preparation for the procedure, the instrument drive unit 20 is coupled to the robotic arm 2 and the housing 36 of the uterine manipulator 10 is coupled to the instrument drive unit 20. Upon coupling the uterine manipulator 10 to the instrument drive unit 20, the drive members 26, 28 of the instrument drive unit 20 are drivingly coupled with the first and second driven members 30, 32 of the uterine manipulator 10.

To articulate the tip member 34 of the uterine manipulator 10, the first and second motors 22, 24 of the instrument drive unit 20 are actuated, whereby the first and second drive members 22, 24 rotate in opposite directions to rotate the first and second driven members 30, 32 of the uterine manipulator 10 in opposite directions. In some aspects, instead of rotating the first and second driven members 30, 32 in opposite directions, the first and second driven members 30, 32 may have opposite thread handedness, e.g., the first driven member 30 may have a right-handed thread whereas the second driven member 32 may have a left-handed thread.

Since the first and second nuts 48, 50 are threadedly coupled to the first and second driven members 30, 32, rotation of the first and second driven members 30, 32 drive a translation of the first and second nuts 48, 50 along the respective first and second driven members 30, 32 in opposite directions from one another. For example, the first nut 48 may translate distally whereas the second nut 50 may translate proximally. As the first nut 48 translates distally, so does the first cable 52, and as the second nut 50 translates proximally, so does the second cable 54. Due to the distal end portion 52b , 54b of the first and second cables 52, 54 being fixed to opposing sides 42a, 42b of the pivot hub 42/tip member 34, the tip member 34 articulates relative to the distal end portion 44 of the shaft 40. When the tip member 34 is disposed within a uterus, articulation of the tip member 34 functions to manipulate the uterus and maintain the uterus in a selected position.

Persons skilled in the art will understand that the devices and methods specifically described herein and illustrated in the accompanying drawings are non-limiting exemplary embodiments. It is envisioned that the elements and features illustrated or described in connection with one exemplary embodiment may be combined with the elements and features of another without departing from the scope of the present disclosure. As well, one skilled in the art will appreciate further features and advantages of the disclosure based on the above-described embodiments. Accordingly, the disclosure is not to be limited by what has been particularly shown and described, except as indicated by the appended claims.

What is claimed is:

1. A robotically-operated uterine manipulator, comprising:
a housing configured to be coupled to an instrument drive unit;
a shaft extending distally from the housing;
a tip hub pivotably coupled to a distal end portion of the shaft; and
an articulation assembly including:
a first driven member rotationally supported in the housing and having a proximal end portion configured to be operably coupled to a first drive member of the instrument drive unit;
a first nut threadedly coupled to the first driven member, such that the first nut translates along the first driven member upon rotation of the first driven member; and
a first cable having a proximal end portion fixed to the first nut, and a distal end portion coupled to the tip hub, wherein the first cable is configured to translate with the first nut and relative to the first driven member in response to a rotation of the first driven member to articulate the tip hub relative to the shaft.

2. The robotically-operated uterine manipulator according to claim 1, wherein the articulation assembly includes:
a second driven member rotationally supported in the housing and having a proximal end portion configured to be operably coupled to a second drive member of the instrument drive unit;
a second nut threadedly coupled to the second driven member, such that the second nut translates along the second driven member upon rotation of the second driven member; and
a second cable having a proximal end portion fixed to the second nut, and a distal end portion, the distal end portion of the first cable being coupled to a first side of the tip hub, and the distal end portion of the second cable being coupled to a second side of the tip hub.

3. The robotically-operated uterine manipulator according to claim 1, further comprising a rotatable collar, wherein the first driven member has a distal end portion operably coupled to the collar, such that a rotation of the collar rotates the first driven member.

4. The robotically-operated uterine manipulator according to claim 3, wherein the collar has a plurality of gear teeth extending from an inner periphery thereof, and the distal end portion of the first driven member has a gear in meshing engagement with the plurality of gear teeth of the collar.

5. The robotically-operated uterine manipulator according to claim 1, wherein the proximal end portion of the first driven member has an input coupler configured to be non-rotatably coupled to an output coupler of the instrument drive unit.

6. The robotically-operated uterine manipulator according to claim 5, wherein the input coupler is accessible from outside of the housing.

7. The robotically-operated uterine manipulator according to claim 1, wherein the first driven member is parallel with and offset from a longitudinal axis defined by the shaft.

8. A robotic surgical assembly, comprising:
an instrument drive unit including a first motor and a first drive member drivingly coupled to the first motor; and
a robotically-operated uterine manipulator including:
a housing configured to be coupled to the instrument drive unit;
a shaft extending distally from the housing;
a tip hub pivotably coupled to a distal end portion of the shaft;
a tip member configured to be attached to the tip hub; and
an articulation assembly including:
a first driven member rotationally supported in the housing and having a proximal end portion configured to be operably coupled to the first drive member of the instrument drive unit;
a first nut threadedly coupled to the first driven member, such that the first nut translates along the first driven member upon rotation of the first driven member; and
a first cable having a proximal end portion fixed to the first nut, and a distal end portion coupled to the tip hub, wherein the first cable is configured to translate with the first nut and relative to the first driven member in response to a rotation of the first driven member to articulate the tip member relative to the shaft.

9. The robotic surgical assembly according to claim 8, wherein the articulation assembly includes:
a second driven member rotationally supported in the housing and having a proximal end portion configured to be operably coupled to a second drive member of the instrument drive unit;
a second nut threadedly coupled to the second driven member, such that the second nut translates along the second driven member upon rotation of the second driven member; and
a second cable having a proximal end portion fixed to the second nut, and a distal end portion, the distal end portion of the first cable being coupled to a first side of the tip hub, and the distal end portion of the second cable being coupled to a second side of the tip hub.

10. The robotic surgical assembly according to claim 8, wherein the uterine manipulator includes a manually-rotatable collar, and the first driven member has a distal end portion operably coupled to the collar, such that a rotation of the collar rotates the first driven member.

11. The robotic surgical assembly according to claim 10, wherein the collar has a plurality of gear teeth extending from an inner periphery thereof, and the distal end portion of the first driven member has a gear in meshing engagement with the plurality of gear teeth of the collar.

12. The robotic surgical assembly according to claim 8, wherein the proximal end portion of the first driven member has an input coupler configured to be non-rotatably coupled to an output coupler of the first drive member.

13. The robotic surgical assembly according to claim 12, wherein the input coupler is accessible from outside of the housing.

14. The robotic surgical assembly according to claim 8, wherein the first driven member is parallel with and offset from a longitudinal axis defined by the shaft.

15. The robotic surgical assembly according to claim 8, further comprising a robotic arm configured to support the instrument drive unit and the uterine manipulator.

16. A robotically-operated uterine manipulator, comprising:
a housing configured to be coupled to an instrument drive unit;
a shaft extending distally from the housing;
a tip hub pivotably coupled to a distal end portion of the shaft;
first and second driven members rotationally supported in the housing and each having a proximal end portion configured to be operably coupled to a respective drive member of the instrument drive unit;
first and second nuts threadedly coupled to the respective first and second driven members, such that the first and second nuts are independently movable along the respective first and second driven members upon rotation of the first and second driven members; and
first and second cables each having a proximal end portion coupled to the respective first and second nuts, and a distal end portion coupled to the tip hub, wherein the first and second cables are configured to translate in opposite directions from one another in response to a rotation of the first and second driven members to articulate the tip hub relative to the shaft.

17. The robotically-operated uterine manipulator according to claim 16, further comprising a rotatable collar, wherein each of the first and second driven members has a distal end portion operably coupled to the collar, such that a rotation of the collar rotates the first and second driven members to translate the first and second cables in opposite directions from one another.

18. The robotically-operated uterine manipulator according to claim 17, wherein the collar has a plurality of gear teeth extending from an inner periphery thereof, and the distal end portion of each of the first and second driven members has a gear in meshing engagement with the plurality of gear teeth of the collar.

19. The robotically-operated uterine manipulator according to claim 16, wherein the proximal end portion of each of the first and second driven members has an input coupler configured to be non-rotatably coupled to a corresponding output coupler of the instrument drive unit.

20. The robotically-operated uterine manipulator according to claim 16, wherein the first and second driven members are parallel with and offset from a longitudinal axis defined by the shaft.
